SEQUENCE LISTING (74 sequences)

SEQ ID NO: 1 - protein, 95 aa, synthetic construct
MPPSNLRVTDVTSTSVTLSWEPPEDGGGPITGYIVEYREADGSGEWQEVTVPGSETSYTVTGLEPGTEYEFRVRAVNGAGEGPPSEPSPVTTPEP

SEQ ID NO: 2 - protein, 95 aa, synthetic construct
MPPSNLRVTDVTSTSVTLSWEPPEDGGGPITGYIVEYREADGSGEWQEVTVPGSETSYTVTGLEPGTEYEFRVRAVNGAGEGPPSEPVPVTTPEP

SEQ ID NO: 3 - protein, 95 aa, synthetic construct
MPPSNLRVTDVTSTSVTLSWEPPEDGGGPITGYIVEYREADGSGEWQEVNVPGSETSYTVTGLEPGTEYEFRVRAVNGAGEGPPSEPSPVTTPEP

SEQ ID NO: 4 - protein, 95 aa, synthetic construct
MPPSNLRVTDVTSTSVTLSWEPPEDGGGPITGYIVEYREADGSGEWQEVNVPGSETSYTVTGLEPGTEYEFRVRAVNGAGEGPPSEPVPVTTPEP

SEQ ID NO: 5 - protein, 94 aa, synthetic construct
MPPSNLRVTDVTSTSVTLSWEPPEDGGPITGYIVEYREADGSGEWQEVTVPGSETSYTVTGLEPGTEYEFRVRAVNGAGEGPPSEPVPVTTPEP

SEQ ID NO: 6 - protein, 93 aa, synthetic construct
MPPSNLRVTDVTSTSVTLSWEPPEGGPITGYIVEYREADGSGEWQEVTVPGSETSYTVTGLEPGTEYEFRVRAVNGAGEGPPSEPVPVTTPEP

SEQ ID NO: 7 - protein, 93 aa, synthetic construct
MPPSNLRVTDVTSTSVTLSWEPPGGGPITGYIVEYREADGSGEWQEVTVPGSETSYTVTGLEPGTEYEFRVRAVNGAGEGPPSEPVPVTTPEP

SEQ ID NO: 8 - protein, 92 aa, synthetic construct
MPPSNLRVTDVTSTSVTLSWEPPGGPITGYIVEYREADGSGEWQEVTVPGSETSYTVTGLEPGTEYEFRVRAVNGAGEGPPSEPVPVTTPEP

SEQ ID NO: 9 - protein, 91 aa, synthetic construct
MPPSNLRVTDVTSTSVTLSWEPPGPITGYIVEYREADGSGEWQEVTVPGSETSYTVTGLEPGTEYEFRVRAVNGAGEGPPSEPVPVTTPEP

SEQ ID NO: 10 - protein, 90 aa, synthetic construct
MPPSNLRVTDVTSTSVTLSWEPGPITGYIVEYREADGSGEWQEVTVPGSETSYTVTGLEPGTEYEFRVRAVNGAGEGPPSEPVPVTTPEP

SEQ ID NO: 11 - protein, 94 aa, synthetic construct
MPPSNLRVTDVTSTSVTLSWEPPEDGGGPITGYIVEYREAGSGEWQEVTVPGSETSYTVTGLEPGTEYEFRVRAVNGAGEGPPSEPVPVTTPEP

SEQ ID NO: 12 - protein, 93 aa, synthetic construct
MPPSNLRVTDVTSTSVTLSWEPPEDGGGPITGYIVEYREASGEWQEVTVPGSETSYTVTGLEPGTEYEFRVRAVNGAGEGPPSEPVPVTTPEP

SEQ ID NO: 13 - protein, 92 aa, synthetic construct
MPPSNLRVTDVTSTSVTLSWEPPEDGGGPITGYIVEYRESGEWQEVTVPGSETSYTVTGLEPGTEYEFRVRAVNGAGEGPPSEPVPVTTPEP

SEQ ID NO: 14 - protein, 97 aa, synthetic construct
MLSPPSNLRVTDVTSTSVTLSWEPPEDGGGPITGYIVEYREADGSGEWQEVTVPGSETSYTVTGLEPGTEYEFRVRAVNGAGEGPPSEPVPVTTPEP

SEQ ID NO: 15 - protein, 97 aa, synthetic construct
MSAPPSNLRVTDVTSTSVTLSWEPPEDGGGPITGYIVEYREADGSGEWQEVTVPGSETSYTVTGLEPGTEYEFRVRAVNGAGEGPPSEPVPVTTPEP

SEQ ID NO: 16 - protein, 94 aa, synthetic construct
MPPSNLRVTDVTSTSVTLSWEPPEDGGGPITGYIVEYREADGSGEWQEVTVPGSETSYTVTGLEPGTEYEFRVRAVNGAGEGPPSEVTVTTPEP

SEQ ID NO: 17 - protein, 95 aa, synthetic construct
MPPSNLRVTDVTSTSVTLSWEPPEDGGGPITGYIVEYREADGSGEWQEVTVPGSETSYTVTGLEPGTEYEFRVRAVNGAGEGPPSESVTVTTPEP

SEQ ID NO: 18 - protein, 97 aa, synthetic construct
MLSPPSNLRVTDVTSTSVTLSWEPPEDGGGPITGYIVEYREADGSGEWQEVTVPGSETSYTVTGLEPGTEYEFRVRAVNGAGEGPPSESVTVTTPEP

SEQ ID NO: 19 - protein, 97 aa, synthetic construct
MSAPPSNLRVTDVTSTSVTLSWEPPEDGGGPITGYIVEYREADGSGEWQEVTVPGSETSYTVTGLEPGTEYEFRVRAVNGAGEGPPSESVTVTTPEP

SEQ ID NO: 20 - protein, 93 aa, synthetic construct
MLSPPSNLRVTDVTSTSVTLSWEPPEPITGYIVEYREADGSGEWQEVTVPGSETSYTVTGLEPGTEYEFRVRAVNGAGEGPPSESVTVTTPEP

SEQ ID NO: 21 - protein, 90 aa, synthetic construct
MPPSNLRVTDVTSTSVTLSWEPPEPITGYIVEYREADGSGEWQEVTVPGSETSYTVTGLEPGTEYEFRVRAVNGAGEGPPSEVTVTTPEP

SEQ ID NO: 22 - protein, 93 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSQSVTVTTPEP

SEQ ID NO: 23 - protein, 97 aa, synthetic construct
MLSPPSNLRVTDVTSTSVTLSWKPPADGGGPITGYIVEYREKDGSGEWKEVTVPGSETSYTVTGLKPGTEYEFRVRAVNGAGEGPPSQSVTVTTPEP

SEQ ID NO: 24 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSQSVTVTT

SEQ ID NO: 25 - protein, 90 aa, synthetic construct
MLSPPSNLRVTDVTSTSVTLSWKPPAPITGYAVEYREKDGSGEWKEVTVPGSETSYTVTGLKPGTEYEFRVRAVNGAGEGPPSQSVTVTT

SEQ ID NO: 26 - protein, 90 aa, synthetic construct
MLSPPSNLRVTDVTSTSVTLSWKPPAPITGYIVAYREKDGSGEWKEVTVPGSETSYTVTGLKPGTEYEFRVRAVNGAGEGPPSQSVTVTT

SEQ ID NO: 27 - protein, 90 aa, synthetic construct
MLSPPSNLRVTDVTSTSVTLSWKPPAPITGYIVEYAEKDGSGEWKEVTVPGSETSYTVTGLKPGTEYEFRVRAVNGAGEGPPSQSVTVTT

SEQ ID NO: 28 - protein, 90 aa, synthetic construct
MLSPPSNLRVTDVTSTSVTLSWKPPAPITGYIVEYREADGSGEWKEVTVPGSETSYTVTGLKPGTEYEFRVRAVNGAGEGPPSQSVTVTT

SEQ ID NO: 29 - protein, 90 aa, synthetic construct
MLSPPSNLRVTDVTSTSVTLSWKPPAPITGYIVEYREKAGSGEWKEVTVPGSETSYTVTGLKPGTEYEFRVRAVNGAGEGPPSQSVTVTT

SEQ ID NO: 30 - protein, 90 aa, synthetic construct
MLSPPSNLRVTDVTSTSVTLSWKPPAPITGYIVEYREKDASGEWKEVTVPGSETSYTVTGLKPGTEYEFRVRAVNGAGEGPPSQSVTVTT

SEQ ID NO: 31 - protein, 90 aa, synthetic construct
MLSPPSNLRVTDVTSTSVTLSWKPPAPITGYIVEYREKDGAGEWKEVTVPGSETSYTVTGLKPGTEYEFRVRAVNGAGEGPPSQSVTVTT

SEQ ID NO: 32 - protein, 90 aa, synthetic construct
MLSPPSNLRVTDVTSTSVTLSWKPPAPITGYIVEYREKDGSGEWKAVTVPGSETSYTVTGLKPGTEYEFRVRAVNGAGEGPPSQSVTVTT

SEQ ID NO: 33 - protein, 90 aa, synthetic construct
MLSPPSNLRVTDVTSTSVTLSWKPPAPITGYIVEYREKDGSGEWKEVAVPGSETSYTVTGLKPGTEYEFRVRAVNGAGEGPPSQSVTVTT

SEQ ID NO: 34 - protein, 90 aa, synthetic construct
MLSPPSNLRVTDVTSTSVTLSWKPPAPITGYIVEYREKDGSGEWKEVTVPGSETSYTVTGLKPGTEYAFRVRAVNGAGEGPPSQSVTVTT

SEQ ID NO: 35 - protein, 90 aa, synthetic construct
MLSPPSNLRVTDVTSTSVTLSWKPPAPITGYIVEYREKDGSGEWKEVTVPGSETSYTVTGLKPGTEYEFAVRAVNGAGEGPPSQSVTVTT

SEQ ID NO: 36 - protein, 90 aa, synthetic construct
MLSPPSNLRVTDVTSTSVTLSWKPPAPITGYIVEYREKDGSGEWKEVTVPGSETSYTVTGLKPGTEYEFRVAAVNGAGEGPPSQSVTVTT

SEQ ID NO: 37 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAAEGPPSQSVTVTT

SEQ ID NO: 38 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGAGPPSQSVTVTT

SEQ ID NO: 39 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGAPSQSVTVTT

SEQ ID NO: 40 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSASVTVTT

SEQ ID NO: 41 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSQSATVTT

SEQ ID NO: 42 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSQAVTVTT

SEQ ID NO: 43 - protein, 90 aa, synthetic construct
MLSPPSNLRVTDVTSTSVTLSWKPPAPITGYIVEYREKDGSGEWKEVTVPGSETSYTVTGLKPGTEYEFRVRAVNGAGEGPPSQSVAVTT

SEQ ID NO: 44 - protein, 90 aa, synthetic construct
  UNSURE 32: note = The symbol X means any one of the amino acids in the group consisting of A, R, N, D, C, Q, E, G, H, I, L, K, M, F, P, O, S, U, T, W, Y, and V
  UNSURE 34: note = The symbol X means any one of the amino acids in the group consisting of A, R, N, D, C, Q, E, G, H, I, L, K, M, F, P, O, S, U, T, W, Y, and V
  UNSURE 36: note = The symbol X means any one of the amino acids in the group consisting of A, R, N, D, C, Q, E, G, H, I, L, K, M, F, P, O, S, U, T, W, Y, and V
  UNSURE 38: note = The symbol X means any one of the amino acids in the group consisting of A, R, N, D, C, Q, E, G, H, I, L, K, M, F, P, O, S, U, T, W, Y, and V
  UNSURE 39: note = The symbol X means any one of the amino acids in the group consisting of A, R, N, D, C, Q, E, G, H, I, L, K, M, F, P, O, S, U, T, W, Y, and V
  UNSURE 40: note = The symbol X means any one of the amino acids in the group consisting of A, R, N, D, C, Q, E, G, H, I, L, K, M, F, P, O, S, U, T, W, Y, and V
  UNSURE 41: note = The symbol X means any one of the amino acids in the group consisting of A, R, N, D, C, Q, E, G, H, I, L, K, M, F, P, O, S, U, T, W, Y, and V
  UNSURE 46: note = The symbol X means any one of the amino acids in the group consisting of A, R, N, D, C, Q, E, G, H, I, L, K, M, F, P, O, S, U, T, W, Y, and V
  UNSURE 48: note = The symbol X means any one of the amino acids in the group consisting of A, R, N, D, C, Q, E, G, H, I, L, K, M, F, P, O, S, U, T, W, Y, and V
  UNSURE 68: note = The symbol X means any one of the amino acids in the group consisting of A, R, N, D, C, Q, E, G, H, I, L, K, M, F, P, O, S, U, T, W, Y, and V
  UNSURE 72: note = The symbol X means any one of the amino acids in the group consisting of A, R, N, D, C, Q, E, G, H, I, L, K, M, F, P, O, S, U, T, W, Y, and V
  UNSURE 78: note = The symbol X means any one of the amino acids in the group consisting of A, R, N, D, C, Q, E, G, H, I, L, K, M, F, P, O, S, U, T, W, Y, and V
  UNSURE 79: note = The symbol X means any one of the amino acids in the group consisting of A, R, N, D, C, Q, E, G, H, I, L, K, M, F, P, O, S, U, T, W, Y, and V
  UNSURE 81: note = The symbol X means any one of the amino acids in the group consisting of A, R, N, D, C, Q, E, G, H, I, L, K, M, F, P, O, S, U, T, W, Y, and V
  UNSURE 70: note = The symbol X means any one of the amino acids in the group consisting of A, R, N, D, C, Q, E, G, H, I, L, K, M, F, P, O, S, U, T, W, Y, and V
  UNSURE 87: note = The symbol X means any one of the amino acids in the group consisting of A, R, N, D, C, Q, E, G, H, I, L, K, M, F, P, O, S, U, T, W, Y, and V
  UNSURE 85: note = The symbol X means any one of the amino acids in the group consisting of A, R, N, D, C, Q, E, G, H, I, L, K, M, F, P, O, S, U, T, W, Y, and V
MLSPPSNLRVTDVTSTSVTLSWKPPAPITGYXVXYXEXXXXGEWKXVXVPGSETSYTVTGLKPGTEYXFXVXAVNGAXXGXPSQXVXVTT

SEQ ID NO: 45 - protein, 9 aa, synthetic construct
  UNSURE 4: note = The symbol X means any one of the amino acids in the group consisting of A, R, N, D, C, Q, E, G, H, I, L, K, M, F, P, O, S, U, T, W, Y, and V
  UNSURE 6: note = The symbol X means any one of the amino acids in the group consisting of A, R, N, D, C, Q, E, G, H, I, L, K, M, F, P, O, S, U, T, W, Y, and V
  UNSURE 8: note = The symbol X means any one of the amino acids in the group consisting of A, R, N, D, C, Q, E, G, H, I, L, K, M, F, P, O, S, U, T, W, Y, and V
TGYXVXYXE

SEQ ID NO: 46 - skipped

SEQ ID NO: 47 - protein, 7 aa, synthetic construct
  UNSURE 3: note = The symbol X means any one of the amino acids in the group consisting of A, R, N, D, C, Q, E, G, H, I, L, K, M, F, P, O, S, U, T, W, Y, and V
  UNSURE 5: note = The symbol X means any one of the amino acids in the group consisting of A, R, N, D, C, Q, E, G, H, I, L, K, M, F, P, O, S, U, T, W, Y, and V
WKXVXVP

SEQ ID NO: 48 - protein, 10 aa, synthetic construct
  UNSURE 4: note = The symbol X means any one of the amino acids in the group consisting of A, R, N, D, C, Q, E, G, H, I, L, K, M, F, P, O, S, U, T, W, Y, and V
  UNSURE 6: note = The symbol X means any one of the amino acids in the group consisting of A, R, N, D, C, Q, E, G, H, I, L, K, M, F, P, O, S, U, T, W, Y, and V
  UNSURE 8: note = The symbol X means any one of the amino acids in the group consisting of A, R, N, D, C, Q, E, G, H, I, L, K, M, F, P, O, S, U, T, W, Y, and V
TEYXFXVXAV

SEQ ID NO: 49 - protein, 6 aa, synthetic construct
  UNSURE 4: note = The symbol X means any one of the amino acids in the group consisting of A, R, N, D, C, Q, E, G, H, I, L, K, M, F, P, O, S, U, T, W, Y, and V
  UNSURE 5: note = The symbol X means any one of the amino acids in the group consisting of A, R, N, D, C, Q, E, G, H, I, L, K, M, F, P, O, S, U, T, W, Y, and V
NGAXXG

SEQ ID NO: 50 - protein, 10 aa, synthetic construct
  UNSURE 1: note = The symbol X means any one of the amino acids in the group consisting of A, R, N, D, C, Q, E, G, H, I, L, K, M, F, P, O, S, U, T, W, Y, and V
  UNSURE 5: note = The symbol X means any one of the amino acids in the group consisting of A, R, N, D, C, Q, E, G, H, I, L, K, M, F, P, O, S, U, T, W, Y, and V
  UNSURE 7: note = The symbol X means any one of the amino acids in the group consisting of A, R, N, D, C, Q, E, G, H, I, L, K, M, F, P, O, S, U, T, W, Y, and V
XPSQXVXVTT

SEQ ID NO: 51 - protein, 90 aa, synthetic construct
MLSPPSNLRVTDVTSTSVTLSWKPPAPITGYIVFYGESLSEGEWKTVNVPGSETSYTVTGLKPGTEYTFGVDAVNGATFGPPSQWVFVTT

SEQ ID NO: 52 - protein, 90 aa, synthetic construct
MLSPPSNLRVTDVTSTSVTLSWKPPAPITGYAVLYTETYYEGEWKQVHVPGSETSYTVTGLKPGTEYDFRVSAVNGATIGTPSQYVIVTT

SEQ ID NO: 53 - protein, 90 aa, synthetic construct
MLSPPSNLRVTDVTSTSVTLSWKPPAPITGYVVLYWEASVQGEWKWVLVPGSETSYTVTGLKPGTEYTFGVDAVNGATFGPPSQWVFVTT

SEQ ID NO: 54 - protein, 90 aa, synthetic construct
MLSPPSNLRVTDVTSTSVTLSWKPPAPITGYRVGYVEHLGAGEWKFVWVPGSETSYTVTGLKPGTEYTFGVDAVNGATFGPPSQWVFVTT

SEQ ID NO: 55 - protein, 90 aa, synthetic construct
MLSPPSNLRVTDVTSTSVTLSWKPPAPITGYWVKYEEGPEYGEWKTVHVPGSETSYTVTGLKPGTEYTFGVDAVNGATFGPPSQWVFVTT

SEQ ID NO: 56 - protein, 90 aa, synthetic construct
MLSPPSNLRVTDVTSTSVTLSWKPPAPITGYPVHYAESDLKGEWKRVVVPGSETSYTVTGLKPGTEYVFIVQAVNGAVDGNPSQIVVVTT

SEQ ID NO: 57 - protein, 90 aa, synthetic construct
MLSPPSNLRVTDVTSTSVTLSWKPPAPITGYVVVYQETIHPGEWKNVHVPGSETSYTVTGLKPGTEYWFGVDAVNGATFGPPSQWVFVTT

SEQ ID NO: 58 - protein, 90 aa, synthetic construct
MLSPPSNLRVTDVTSTSVTLSWKPPAPITGYWVSYLESTFIGEWKWVHVPGSETSYTVTGLKPGTEYVFVVQAVNGAPFGGPSQHVVVTT

SEQ ID NO: 59 - protein, 90 aa, synthetic construct
MLSPPSNLRVTDVTSTSVTLSWKPPAPITGYIVHYSEFIFVGEWKHVLVPGSETSYTVTGLKPGTEYDFRVSAVNGATIGTPSQYVIVTT

SEQ ID NO: 60 - protein, 90 aa, synthetic construct
MLSPPSNLRVTDVTSTSVTLSWKPPAPITGYFVLYKELLQDGEWKTVLVPGSETSYTVTGLKPGTEYPFPVWAVNGALAGFPSQFVEVTT

SEQ ID NO: 61 - protein, 90 aa, synthetic construct
MLSPPSNLRVTDVTSTSVTLSWKPPAPITGYAVYYNETWFQGEWKHVVVPGSETSYTVTGLKPGTEYYFHVEAVNGANPGKPSQHVVVTT

SEQ ID NO: 62 - protein, 90 aa, synthetic construct
MLSPPSNLRVTDVTSTSVTLSWKPPAPITGYTVGYIEHPLSGEWKIVYVPGSETSYTVTGLKPGTEYTFGVDAVNGATFGPPSQWVFVTT

SEQ ID NO: 63 - protein, 90 aa, synthetic construct
MLSPPSNLRVTDVTSTSVTLSWKPPAPITGYSVTYNETTLDGEWKQVSVPGSETSYTVTGLKPGTEYVFSVNAVNGAKEGEPSQWVVVTT

SEQ ID NO: 64 - protein, 90 aa, synthetic construct
MLSPPSNLRVTDVTSTSVTLSWKPPAPITGYQVVYSESVGWGEWKQVKVPGSETSYTVTGLKPGTEYWFGVDAVNGATFGPPSQWVFVTT

SEQ ID NO: 65 - protein, 90 aa, synthetic construct
MLSPPSNLRVTDVTSTSVTLSWKPPAPITGYGVSYEEVYGFGEWKHVYVPGSETSYTVTGLKPGTEYVFSVLAVNGATFGPPSQWVFVTT

SEQ ID NO: 66 - protein, 90 aa, synthetic construct
MLSPPSNLRVTDVTSTSVTLSWKPPAPITGYYVNYYEGPSDGEWKYVKVPGSETSYTVTGLKPGTEYWFWVQAVNGASVGPPSQDVGVTT

SEQ ID NO: 67 - protein, 90 aa, synthetic construct
MLSPPSNLRVTDVTSTSVTLSWKPPAPITGYYVRYSESVDYGEWKWVLVPGSETSYTVTGLKPGTEYTFGVDAVNGATFGPPSQWVFVTT

SEQ ID NO: 68 - protein, 90 aa, synthetic construct
MLSPPSNLRVTDVTSTSVTLSWKPPAPITGYYVSYEEHPNDGEWKYVEVPGSETSYTVTGLKPGTEYTFGVDAVNGATFGPPSQWVFVTT

SEQ ID NO: 69 - protein, 90 aa, synthetic construct
MLSPPSNLRVTDVTSTSVTLSWKPPAPITGYFVSYTEGLEQGEWKFVLVPGSETSYTVTGLKPGTEYTFGVDAVNGATFGPPSQWVFVTT

SEQ ID NO: 70 - protein, 90 aa, synthetic construct
MLSPPSNLRVTDVTSTSVTLSWKPPAPITGYWVTYEESTSHGEWKFVWVPGSETSYTVTGLKPGTEYTFGVDAVNGATFGPPSQWVFVTT

SEQ ID NO: 71 - protein, 90 aa, synthetic construct
MLSPPSNLRVTDVTSTSVTLSWKPPAPITGYGVLYQESLHDGEWKWVLVPGSETSYTVTGLKPGTEYTFGVDAVNGATFGPPSQWVFVTT

SEQ ID NO: 72 - protein, 90 aa, synthetic construct
MLSPPSNLRVTDVTSTSVTLSWKPPAPITGYYVFYDETVFLGEWKHVYVPGSETSYTVTGLKPGTEYAFTVAAVNGAVQGNPSQGVVVTT

SEQ ID NO: 73 - protein, 90 aa, synthetic construct
MLSPPSNLRVTDVTSTSVTLSWKPPAPITGYYVQYNETTLTGEWKQVRVPGSETSYTVTGLKPGTEYIFNVHAVNGAHYGDPSQVVTVTT

SEQ ID NO: 74 - protein, 89 aa, synthetic construct
  UNSURE 31: note = The symbol X means any one of the amino acids in the group consisting of A, R, N, D, C, Q, E, G, H, I, L, K, M, F, P, O, S, U, T, W, Y, and V
  UNSURE 33: note = The symbol X means any one of the amino acids in the group consisting of A, R, N, D, C, Q, E, G, H, I, L, K, M, F, P, O, S, U, T, W, Y, and V
  UNSURE 35: note = The symbol X means any one of the amino acids in the group consisting of A, R, N, D, C, Q, E, G, H, I, L, K, M, F, P, O, S, U, T, W, Y, and V
  UNSURE 37: note = The symbol X means any one of the amino acids in the group consisting of A, R, N, D, C, Q, E, G, H, I, L, K, M, F, P, O, S, U, T, W, Y, and V
  UNSURE 38: note = The symbol X means any one of the amino acids in the group consisting of A, R, N, D, C, Q, E, G, H, I, L, K, M, F, P, O, S, U, T, W, Y, and V
  UNSURE 39: note = The symbol X means any one of the amino acids in the group consisting of A, R, N, D, C, Q, E, G, H, I, L, K, M, F, P, O, S, U, T, W, Y, and V
  UNSURE 40: note = The symbol X means any one of the amino acids in the group consisting of A, R, N, D, C, Q, E, G, H, I, L, K, M, F, P, O, S, U, T, W, Y, and V
  UNSURE 45: note = The symbol X means any one of the amino acids in the group consisting of A, R, N, D, C, Q, E, G, H, I, L, K, M, F, P, O, S, U, T, W, Y, and V
  UNSURE 47: note = The symbol X means any one of the amino acids in the group consisting of A, R, N, D, C, Q, E, G, H, I, L, K, M, F, P, O, S, U, T, W, Y, and V
  UNSURE 67: note = The symbol X means any one of the amino acids in the group consisting of A, R, N, D, C, Q, E, G, H, I, L, K, M, F, P, O, S, U, T, W, Y, and V
  UNSURE 69: note = The symbol X means any one of the amino acids in the group consisting of A, R, N, D, C, Q, E, G, H, I, L, K, M, F, P, O, S, U, T, W, Y, and V
  UNSURE 71: note = The symbol X means any one of the amino acids in the group consisting of A, R, N, D, C, Q, E, G, H, I, L, K, M, F, P, O, S, U, T, W, Y, and V
  UNSURE 77: note = The symbol X means any one of the amino acids in the group consisting of A, R, N, D, C, Q, E, G, H, I, L, K, M, F, P, O, S, U, T, W, Y, and V
  UNSURE 78: note = The symbol X means any one of the amino acids in the group consisting of A, R, N, D, C, Q, E, G, H, I, L, K, M, F, P, O, S, U, T, W, Y, and V
  UNSURE 80: note = The symbol X means any one of the amino acids in the group consisting of A, R, N, D, C, Q, E, G, H, I, L, K, M, F, P, O, S, U, T, W, Y, and V
  UNSURE 86: note = The symbol X means any one of the amino acids in the group consisting of A, R, N, D, C, Q, E, G, H, I, L, K, M, F, P, O, S, U, T, W, Y, and V
  UNSURE 84: note = The symbol X means any one of the amino acids in the group consisting of A, R, N, D, C, Q, E, G, H, I, L, K, M, F, P, O, S, U, T, W, Y, and V
LSPPSNLRVTDVTSTSVTLSWKPPAPITGYXVXYXEXXXXGEWKXVXVPGSETSYTVTGLKPGTEYXFXVXAVNGAXXGXPSQXVXVTT

(19) United States
(12) Patent Application Publication
(10) Pub. No.: US 2025/0085290 A1
(43) Pub. Date: Mar. 13, 2025

(54) HUMAN FIBRONECTIN TYPE III PROTEIN SCAFFOLDS

(71) Applicant: ARO BIOTHERAPEUTICS COMPANY, Philadelphia, PA (US)

(72) Inventors: Ronald V. Swanson, Philadelphia, PA (US); Zhanna Druzina, Philadelphia, PA (US); Yao Xin, Philadelphia, PA (US); Karyn O’Neil, Philadelphia, PA (US)

(21) Appl. No.: 18/580,205

(22) PCT Filed: Jul. 19, 2022
(86) PCT No.: PCT/US2022/037586

§ 371 Date: Jan. 18, 2024

Related U.S. Application Data
(60) Provisional application No. 63/203,343, filed on Jul. 19, 2021.

(51) Int. Cl. G01N 33/68; C07K 14/78

(52) U.S. Cl. CPC G01N 33/6845; C07K 14/78

(57) ABSTRACT

Protein scaffolds and scaffold libraries based on a fibronectin type III (FN3) domain with an alternative binding surface design, isolated nucleic acids encoding the protein scaffolds, vectors, host cells, methods of making thereof, and uses as therapeutic molecules for treatment and diagnosis of diseases and disorders.

### RELATED APPLICATIONS

The present application claims priority to U.S. Provisional Application No. 63/203,343, filed Jul. 19, 2021, which is hereby incorporated by reference in its entirety.

### FIELD

The present disclosure relates to fibronectin type III (FN3) domain molecules and methods of making and using the molecules.

### BACKGROUND

Monoclonal antibodies are the most widely used class of therapeutic proteins when high affinity and specificity for a target molecule are desired. However, non-antibody proteins having relatively defined three-dimensional structures that can be engineered to bind desired target molecules, commonly referred to as protein scaffolds, may have advantages over traditional antibodies due to their small size, lack of disulfide bonds, high stability, and ability to be expressed in prokaryotic hosts. These scaffolds typically contain one or more regions which are amenable to specific or random sequence variation, and such sequence randomization is often carried out to produce libraries of proteins from which desired products may be selected. Novel methods of purification are readily applied; scaffolds are easily conjugated to drugs/toxins, penetrate efficiently into tissues and can be formatted into multispecific binders (Binz and Pluckthun, Curr Opin Biotechnol, 16, 459-469, 2005; Skerra, J Mal Recognit, 13, 167-187, 2000).

One such protein scaffold is the fibronectin type III (FN3) domain identified in a multitude of proteins, having a characteristic tertiary structure with 6 loops connected by 7 beta strands. Three loops in particular, the FG, BC, and DE loops are structurally analogous to the complementarity determining regions (CDRs) of antibodies. These loops have been randomized to generate libraries of the FN3 domain scaffolds to successfully select specific binders to a number of different targets while retaining important biophysical properties (Getmanova et al., Chem Biol, 13, 549-556, 2006; Hackel et al., J Mol Biol, 381, 1238-1252, 2008; Karatan et al., Chem Biol, 11, 835-844, 2004; Koide et al., J Mol Biol, 284, 1141-1151, 1998; Koide et al., Proc Natl Acad Sci US A, 104, 6632-6637, 2007; Parker et al., Protein Eng Des Sel, 18, 435-444, 2005; Xu et al., Chemistry & Biology, 9, 933-942, 2002).

There is a need for alternative binding domains, which can be used as targeting moieties that can facilitate the delivery of a therapeutic, such as an oligonucleotide based therapeutic, or have a direct therapeutic effect by binding to a target molecule. The present disclosure provides such improved proteins.

### SUMMARY

In some embodiments, a library comprising a plurality of fibronectin type III module (FN3) domains (polypeptides) is provided, the library having a diversified C-CD-D-F-FG-G alternative surface comprising a diversified C beta-strand, a CD loop, a D beta-strand, an F beta-strand, an FG loop and a G beta-strand, wherein the polypeptides comprise an amino acid sequence at least 80%, 85%, 90%, or 95% identical to the amino acid sequence of SEQ ID NO: 44; wherein the plurality of polypeptides comprises at least one mutated amino acid residue as compared to SEQ ID NO: 24 in one or more of, or each of, the C beta-strand, the CD loop, the D beta-strand, the F beta-strand, the FG loop, or the G beta-strand to form the FN3 domain library having the diversified C-CD-D-F-FG-G alternative surface.

In some embodiments, a method of producing the library described herein is provided.

In some embodiments, methods of making a library of human fibronectin type III (FN3) domains are provided, wherein the library comprises a diversified C-CD-D-F-FG-G alternative surface comprising a diversified one or more, or each of, C beta-strand, a CD loop, a D beta-strand, an F beta-strand, an FG loop, and G beta-strand comprising providing a reference FN3 domain polypeptide having the amino acid sequence at least 80, 85, or 90% identical to that of SEQ ID NO: 44; introducing diversity into the reference FN3 domain polypeptide by mutating at least one residue of any one of the following domains: C beta-strand, a CD loop, a D beta-strand, an F beta-strand, an FG loop, and a G beta-strand residue to form the human FN3 domain library having the diversified C-CD-D-F-FG-G alternative surface.

In some embodiments, a library produced by the methods described herein is provided.

In some embodiments, a method of obtaining a protein scaffold comprising a human fibronectin type III module (FN3) domain having a diversified C-CD-D-F-FG-G alternative surface that specifically binds to a target molecule is provided, the method comprising contacting or panning the library with the target molecule and isolating a protein scaffold specifically binding to the target molecule with a predefined affinity.

In some embodiments, a method of obtaining a polypeptide comprising a fibronectin type III module (FN3) domain having a diversified C-CD-D-F-FG-G alternative surface that binds or specifically binds to a target molecule is provided, the method comprising contacting or panning (screening) a library disclosed herein with the target molecule and isolating the polypeptide that binds or specifically binds to the target molecule.

### DETAILED DESCRIPTION

The term “fibronectin type III (FN3) domain” (FN3 domain) as used herein refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3rd FN3 domain of tenascin (TN3), or the 10th FN3 domain of fibronectin (FN10).

The term “alternative surface” as used herein refers to a surface on a side of the FN3 domain comprising one or more beta strands, and one or more loop. In some embodiments, alternative surfaces are a C-CD-D-F-FG-G surface that is formed by amino acids in the C beta-strand, the CD loop, the D beta-strand, the F beta-strand, the FG loop, and the G beta-strand. In some embodiments, alternative surfaces comprise a diversified C beta-strand, a CD loop, a D beta-strand, an F beta-strand, an FG loop and a G beta-strand.

The term “biological sample” refers to blood, tissue, marrow, sputum and the like.

The term “diagnostic reagent” refers to any substance that may be used to analyze a biological sample, whether or not such substance is distributed as a single substance or in a combination with other substances in a diagnostic kit.

The term “substituting” or “substituted” or ‘mutating” or “mutated” as used herein refers to altering, deleting of inserting one or more amino acids or nucleotides in a polypeptide or polynucleotide sequence to generate a variant of that sequence.

The term “randomizing” or “randomized” or “diversified” or “diversifying” as used herein refers to making at least one substitution, insertion or deletion in a polynucleotide or polypeptide sequence.

“Variant” as used herein refers to a polypeptide or a polynucleotide that differs from a reference polypeptide or a reference polynucleotide by one or more modifications for example, substitutions, insertions or deletions.

The term “specifically binds” or “specific binding” as used herein refers to the ability of FN3 domain described herein to bind to a predetermined antigen with a dissociation constant (KD) of about 1×10-6 M or less, for example about 1×10-7 M or less, about 1×10-8 M or less, about 1×10-9 M or less, about 1×10-10 M or less, about 1×10-11 M or less, about 1×10-12 M or less, or about 1×10-13 M or less. Typically the FN3 domain binds to a predetermined antigen (i.e. human PSMA) with a KD that is at least ten fold less than its KD for a nonspecific antigen (for example BSA or casein) as measured by surface plasmon resonance using for example a Proteon Instrument (BioRad). The isolated FN3 domain that specifically binds to human PSMA may, however, have cross-reactivity to other related antigens, for example to the same predetermined antigen from other species (homologs), such as Macaca fascicularis (cynomolgous monkey, cyno) or Pan troglodytes (chimpanzee).

The term “target molecule” as used herein refers to a protein, peptide, carbohydrate, lipid, and the like having an antigen or an epitope that is recognized by a FN3 domain. The target molecule may be naturally or non-naturally occurring.

The term “epitope” as used herein means a portion of an antigen to which an FN3 domain specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The term “library” refers to a collection of variants. The library may be composed of polypeptide or polynucleotide variants.

The term “stability” as used herein refers to the ability of a molecule to maintain a folded state under physiological conditions such that it retains at least one of its normal functional activities, for example, binding to a predetermined antigen.

“Tencon” as used herein refers to the synthetic fibronectin type III (FN3) domain having the sequence described in U.S. Pat. Publ. No. US2010/0216708.

The term “tenascin C” as used herein refers to human tenascin C having a sequence shown in GenBank Acc. No. NP_002151. Tenascin Chas 15 tandem FN3 domains.

A “cancer cell” or a “tumor cell” as used herein refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

“Inhibits growth” (e.g. referring to cells, such as tumor cells) refers to a measurable decrease in the cell growth in vitro or in vivo when contacted with a therapeutic or a combination of therapeutics or drugs when compared to the growth of the same cells grown in appropriate control conditions well known to the skilled in the art. Inhibition of growth of a cell in vitro or in vivo may be at least about 10%, 20%, 30%, 40%, 50%, 60%, 70%, 80%, 90%, 99%, or 100%. Inhibition of cell growth may occur by a variety of mechanisms, for example by apoptosis, necrosis, or by inhibition of cell proliferation, or lysis of cells.

The term “vector” means a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

The term “expression vector” means a vector that can be utilized in a biological system or in a reconstituted biological system to direct the translation of a polypeptide encoded by a polynucleotide sequence present in the expression vector.

The term “polynucleotide” means a molecule comprising a chain of nucleotides covalently linked by a sugar-phosphate backbone or other equivalent covalent chemistry. Double and single-stranded DNAs and RNAs are typical examples of polynucleotides.

The term “polypeptide” or “protein” means a molecule that comprises at least two amino acid residues linked by a peptide bond to form a polypeptide. Small polypeptides of less than about 50 amino acids may be referred to as “peptides”.

The term “in combination with” as used herein means that two or more therapeutics can be administered to a subject together in a mixture, concurrently as single agents or sequentially as single agents in any order.

The term “heterologous” means that the polypeptide described has been derived from different cell types or different species and does not exist in nature.

The present embodiments provide FN3 domains that specifically bind to a target molecule, and thus can be widely used in therapeutic and diagnostic applications. The FN3 polypeptides have domains comprising one or more beta-strands and one or more loop, which can be randomized to generate protein scaffolds and select for protein scaffolds specifically binding a target molecule with high affinity. Published FN3-based domain libraries have been generated by diversifying either the top or the bottom loops, areas that structurally resemble CDRs in antibody variable chains, providing curved binding surfaces. In contrast, high affinity binding molecules can be selected from FN3 domain libraries provided for herein that display concave interaction surfaces, which are generated by randomizing an alternative surface as provided herein based on a reference sequence. This can be done, for example, to increase the number of epitopes and targets against which high affinity binding protein scaffolds can be selected. In some embodiments, polynucleotides encoding the protein domains or complementary nucleic acids thereof are provided, vectors, host cells, and methods of making and using them. The present embodiments also provides methods of making libraries of FN3 domains as provided for herein, and libraries made up of the foregoing.

### Fibronectin Type III Domain

The Fibronectin Type III (FN3) domain (or module) is a prototypic repeat domain initially identified in fibronectin and now known to be present in various animal protein families including cell surface receptors, extracellular matrix proteins, enzymes, and muscle proteins. Structurally the FN3 domains have a topology very similar to that of immunoglobulin-like domains, except for the lack of disulfide bonds. As is known in the art, naturally occurring FN3 domains have a beta-sandwich structure having seven beta-strands, referred to as A, B, C, D, E, F, and G, linked by six loops, referred to as AB, BC, CD, DE, EF, and FG loops (Bork and Doolittle, Proc Natl Acad Sci USA 89, 8990-8992, 1992; U.S. Pat. No. 6,673,901). Three loops, the BC, DE and FG loops are at the top of the FN3 domain, and three, the AB, CD and EF loops at the bottom of the domain. While FN3 domain conformations are highly conserved, the similarity between different domains at the amino acid level is quite low. FN3 domains may be naturally or non-naturally occurring. Exemplary non-naturally occurring FN3 domains are a consensus FN3 domain designed based on an alignment of select FN3 domains present in a certain protein and incorporating the most conserved (frequent) amino acid at each position to generate the non-naturally occurring FN3 domain. For example, a non-naturally occurring FN3 domain is designed based on a consensus sequence of the 15 FN3 domains from human tenascin C, or based on a consensus sequence of the 15 FN3 domains from human fibronectin. These non-naturally occurring FN3 domains retain the typical topology of the FN3 domains, and can exhibit improved properties such as improved stability when compared to the wild type FN3 domains. Exemplary non-naturally occurring FN3 domains are the Tencon and the Fibcon domains described in U.S. Pat. Pub. No. 2010/0216708 and U.S. Pat. Pub. No. 2010/0255056. However, there is still a need for improved binding molecules.

Amino acid residues defining each loop and each beta-strand are shown in Table 1 for the FN3 scaffolds described herein. The residues shown below for each domain/region can be determined for another sequence by aligning the two sequences, one the reference sequence of SEQ ID NO: 44, the other being the query sequence. They can be aligned using, for example, Blastp (available through NBCI) to align two sequences, using default settings.

TABLE 1

| FN3 | |
|---|---|
| domain/region | SEQ ID NO: 44 |
| A strand | 1-13 |
| AB loop | 14-17 |
| B strand | 18-22 |
| BC loop | 23-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-80 |
| G strand | 81-90 |

The variability in the FN3 domains to create a library or another sequence can be done in one or more of the following regions: C beta-strand, a CD loop, a D beta-strand, an F beta-strand, an FG loop and a G beta-strand, which can be referred to as a “C-CD-D-F-FG-G alternative surface.”

This alternative surface can be diversified based on a consensus sequence and mutation residues at specific positions to generate a library of polypeptides that can be used to bind target moieties, such as cell surface proteins or receptors or other target molecules. In some embodiments, the library comprises a plurality of proteins that is based on the consensus sequence of SEQ ID NO: 44:

(SEQ ID NO: 44)
MLSPPSNLRVTDVTSTSVTLSWKPPAPITGYXVXYXEXXXXGEW
KXVXVPGSETSYTVTGLKPGTEYXFXVXAVNGAXXGXPSQXVXV
TT

wherein each X is, independently, any amino acid. In some embodiments, each X is, independently, any amino acid, except a methionine or a cysteine.

In some embodiments, the lead methionine of SEQ ID NO: 44 can be removed. Thus, in some embodiments the library comprises a plurality of proteins that is based on the consensus sequence of SEQ ID NO: 74:

(SEQ ID NO: 74)
LSPPSNLRVTDVTSTSVTLSWKPPAPITGYXVXYXEXXXXGE
WKXVXVPGSETSYTVTGLKPGTEYXFXVXAVNGAXXGXPSQX
VXVTT

wherein each X is, independently, any amino acid. In some embodiments, each X is independently, any amino acid, except a methionine or a cysteine.

The alternative surfaces can be described herein in the FN3 domains are encoded by non-contiguous stretches of amino acids in each FN3 domain. For example, the C-CD-D-F-FG-G surface is formed by amino acid residues 29-37, 38-43, 44-50, 65-74, 75-80, and 81-90 of SEQ ID NO: 44, and, for example, as shown in Table 2. In some embodiments, the C-CD-D-F-FG-G surface comprises amino acid residues 29-37, 38-43, 44-50, 65-74, 75-80, and 81-90 of SEQ ID NO: 44.

In some embodiments, a polypeptide is provided herein, the polypeptide comprising an amino acid sequence of SEQ ID NO: 44, wherein each X in SEQ ID NO: 44, is, independently, any amino acid. In some embodiments, each X of SEQ ID NO: 44 is independently, any amino acid, except a methionine or a cysteine.

In some embodiments, a polypeptide is provided herein, the polypeptide comprising an amino acid sequence of SEQ ID NO: 74, wherein each X in SEQ ID NO: 74, is, independently, any amino acid. In some embodiments, each X of SEQ ID NO: 74 is independently, any amino acid, except a methionine or a cysteine.

### Protein Scaffolds Based on Randomizing Alternative Surfaces

In some embodiments, an isolated protein scaffold comprising an FN3 domain comprising an alternative surface, wherein the alternative surface has at least one amino acid substitution in a region of the C-CD-D-F-FG-G alternative surface forming the alternative surface.

In some embodiments, the library comprises a protein or plurality of proteins that are at least, or about, 80%, 85%, 90%, 95%, 96%, 97%, 98%, or 99% to the amino acid sequence of SEQ ID NO: 44.

In some embodiments, the FN3 domain comprises an amino acid sequence that is at least, or about, 80%, 85%, 90%, 95%, 96%, 97%, 98%, or 99% identical to SEQ ID NO: 24.

In some embodiment, the FN3 domain comprises the amino acid sequence of SEQ ID NO: 24.

(SEQ ID NO: 24)
MLSPPSNLRVTDVTSTSVTLSWKPPAPITGYIVEYREKDGSGEWKEVTV
PGSETSYTVTGLKPGTEYEFRVRAVNGAGEGPPSQSVTVTT

In some embodiments, the FN3 domain comprises an amino acid sequence having one or more substitutions at positions 32, 34, 36, 38, 39, 40, 41, 46, 48, 68, 70, 72, 78, 79, 81, 85, and/or 87 of SEQ ID NO: 24. These positions correspond to the domains as illustrated in Table 2 below that can be mutated to create a new FN3 polypeptide or a library of polypeptides.

In some embodiments, the protein scaffold or library comprises a C-CD-D-F-FG-G alternative surface formed by a C beta-strand, a CD loop, a D beta-strand, an F beta-strand, a FG loop, and a G beta-strand. In some embodiments, the protein scaffold or library comprises a C-CD-D-F-FG-G alternative surface that comprises a C beta-strand, a CD loop, a D beta-strand, an F beta-strand, a FG loop, and a G beta-strand. In some embodiments, the protein scaffold or library comprises a C-CD-D-F-FG-G alternative surface that comprises a diversified C beta-strand, CD loop, D beta-strand, F beta-strand, FG loop, and/or beta-strand.

In some embodiments, the C beta-strand, the CD loop, the D beta-strand, the F beta-strand, the FG loop, or the G beta-strand forming the C-CD-D-F-FG-G alternative surface comprise certain amino acid sequences as shown in Table 2 and in SEQ ID NOS: 45-48.

TABLE 2

| | SEQ ID | Amino Acid | SEQ |
|---|---|---|---|
| FN3 domain | NO: 44 | Sequence | ID NO: |
| C strand | 29-37 | TGYXVXYXE | 45 |
| CD loop | 38-43 | XXXXGE | 46 |
| D strand | 44-50 | WKXVXVP | 47 |
| F strand | 65-74 | TEYXFXVXAV | 48 |
| FG loop | 75-80 | NGAXXG | 49 |
| G strand | 81-90 | XPSQXVXVTT | 50 |

wherein each X, is independently, any amino acid, In some embodiments, each X, is independently, any amino acid, except for methionine or cysteine.

In some embodiments, the FN3 domain comprises the C beta-strand having an amino acid sequence TGYXVXYXE (SEQ ID NO: 45) having substitutions at 1, 2, or 3 residues, wherein each X is, independently, any amino acid except a methionine or a cysteine.

In some embodiments, the FN3 domain comprises a CD loop having an amino acid sequence of XXXXGE (SEQ ID NO: 46) having substitutions at 1, 2, 3, or 4 residues, wherein each X is, independently, any amino acid except a methionine or a cysteine.

In some embodiments, the FN3 domain comprises the D beta-strand having an amino acid sequence WKXVXVP (SEQ ID NO: 47) having substitutions at 1, or 2 residues, wherein each X is, independently, any amino acid except a methionine or a cysteine.

In some embodiments, the FN3 domain comprises the F beta-strand having an amino acid sequence TEYXFXVXAV (SEQ ID NO: 48) having substitutions at 1, 2, or 3 residues, wherein each X is, independently, any amino acid except a methionine or a cysteine.

In some embodiments, the FN3 domain comprises the FG loop having an amino acid sequence NGAXXG (SEQ ID NO: 49) having substitutions at 1, or 2 residues, wherein each X is, independently, any amino acid except a methionine or a cysteine.

In some embodiments, the FN3 domain comprises the F beta-strand having an amino acid sequence XPSQXVXVTT (SEQ ID NO: 50) having substitutions at 1, 2, or 3 residues, wherein each X is, independently, any amino acid except a methionine or a cysteine.

In some embodiments, the library comprises a plurality of polypeptides, comprising a sequence of TGYXVXYXE (SEQ ID NO: 45), XXXXGE (SEQ ID NO: 46), WKXVXVP (SEQ ID NO: 47), TEYXFXVXAV (SEQ ID NO: 48), NGAXXG (SEQ ID NO: 49), and XPSQXVXVTT (SEQ ID NO: 50), wherein each X is, independently, any amino acid except a methionine or a cysteine.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 1.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 2.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 3.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 4.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 5.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 6.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 7.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 8.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 9.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 10.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 11.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 12.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 13.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 14.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 15.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 16.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 17.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 18.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 19.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 20.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 21.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 22.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 23.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 24.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 25.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 26.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 27.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 28.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 29.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 30.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 31.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 32.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 33.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 34.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 35.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 36.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 37.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 38.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 39.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 40.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 41.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 42.

In some embodiments, the library comprises a polypeptide or a plurality of polypeptides that comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence of SEQ ID NO: 43.

In some embodiments, polypeptides are provided herein. In some embodiments, the polypeptide comprises an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical, to a sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43. In some embodiments, pharmaceutical compositions comprising the polypeptide are provided.

In some embodiments, the resulting FN3 domains that are based on a consensus or reference sequence provided for herein can be further modified at residues residing outside of or within the alternative surface, such as those provided for herein, for the purpose of for example improving stability, reducing immunogenicity, enhancing binding affinity, on-rate, off-rate, half-life, solubility, or any other suitable characteristics. In one way to achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, CA). Inspection of these displays permits analysis of the likely role of the residues in the functioning of the candidate sequence, for example, residues that influence stability of the scaffold protein or the ability of the candidate scaffold protein to bind its target molecule. In this way, residues can be selected and combined from the parent and reference sequences so that the desired characteristics, such as improved scaffold stability is achieved. Alternatively, or in addition to the above procedures, other suitable methods of engineering can be used as known in the art.

Desirable physical properties of FN3 domains include high thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr Opin Biotechnol, 12, 371-375, 2001). High thermal stability may increase the yield of the expressed protein, improve solubility or activity, decrease immunogenicity, and minimize the need of a cold chain in manufacturing.

Residues that can be substituted to improve any characteristics of the FN3 domains can be determined by making the substitution and assaying for the desired characteristics of the scaffold.

In terms of loss of stability, i.e., “denaturing” or “denaturation” of a protein, is meant the process where some or all of the three-dimensional conformation imparting the functional properties of the protein has been lost with an attendant loss of activity and/or solubility. Forces disrupted during denaturation include intramolecular bonds, for example, electrostatic, hydrophobic, Van der Waals forces, hydrogen bonds, and disulfides. Protein denaturation can be caused by forces applied to the protein or a solution comprising the protein, such as mechanical force (for example, compressive or shear-force), thermal, osmotic stress, change in pH, electrical or magnetic fields, ionizing radiation, ultraviolet radiation and dehydration, and by chemical denaturants.

Measurement of protein stability and protein !ability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“TM”) temperature, the temperature in ° Celsius (0 C) at which ½ of the molecules become unfolded, using standard methods. Typically, the higher the TM, the more stable the molecule. In addition to heat, the chemical environment also changes the ability of the protein to maintain a particular three dimensional structure.

In some embodiments, the FN3 domains exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the TM.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH3COOH), halogenated acetic acids), hydrophobic molecules (e.g. phospholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

In some embodiments, the polypeptides exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same scaffold prior to engineering measured by using guanidinium hydrochloride as a chemical denaturant. Increased stability can be measured as a function of decreased tryptophan fluorescence upon treatment with increasing concentrations of guanidine hydrochloride using well known methods.

The FN3 domains described herein may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. The use of naturally occurring as well as artificial peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456).

The FN3 domains may be used as bispecific molecules wherein the first alternative surface in a domain has specificity for a first target molecule and the second alternative surface in the same domain has specificity for a second target molecule.

The FN3 domains may incorporate other subunits for example via covalent interaction. All or a portion of an antibody constant region may be attached to the FN3 domain to impart antibody-like properties, especially those properties associated with the Fe region, e.g., complement activity, half-life, etc. For example, Fe effector functions such as Clq binding, complement dependent cytotoxicity (CDC), Fe receptor binding, antibody-dependent cell-mediated cytotoxicity (ADCC), phagocytosis, down regulation of cell surface receptors (e.g., B cell receptor; BCR), etc. can be provided and/or controlled by modifying residues in the Fe responsible for these activities (for review; see Strohl, Curr Opin Biotechnol. 20, 685-691, 2009).

Additional moieties may be incorporated into, or conjugated with, the FN3 domains such as toxin conjugates, albumin or albumin binders, polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein coding sequences and may be generated by standard cloning and expression techniques. Alternatively, well known chemical coupling methods may be used to attach the moieties to recombinantly produce FN3 domains described herein. In some embodiments, the FN3 is conjugated with a nucleic acid molecule, such as an antisense molecule, siRNA, PMO, and the like.

FN3 domains incorporating additional moieties may be compared for functionality by several well-known assays. For example, altered FN3 domain properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating protein scaffold pharmacokinetic properties in in vivo models

### Generation and Production of FN3 Domain Proteins

In some embodiments, methods of making a library of FN3 domains comprising an alternative surface, wherein the alternative surface has at least one amino acid substitution when compared to a reference FN3 domain, such as provided for herein are provided. In some embodiments, the methods comprise providing a polynucleotide encoding a reference FN3 domain; generating a library of polynucleotide sequences of the reference FN3 domain by randomizing the alternative surface; translating the library in vitro or expressing the library in a host.

In some embodiments, methods of making a library of FN3 polypeptides having a diversified C-CD-D-F-FG-G alternative surface formed by the C beta-strand, the CD loop, the D beta-strand, the F beta-strand, the FG loop, or the G beta-strand are provided, the method comprising providing a reference FN3 domain polypeptide having the amino acid sequence at least, or about, 80%, 81%, 82%, 83%, 84%, 85%, 86%, 87%, 88%, 89%, 90%, 91%, 92%, 93%, 94%, 95%, 96%, 97%, 98%, 99%, identical to that of SEQ ID NO: 44; introducing diversity into the consensus FN3 domain polypeptide by mutating at least one residue in the C beta-strand, the CD loop, the D beta-strand, the F beta-strand, the FG loop, or the G beta-strand to form the FN3 domain library having the diversified C-CD-D-F-FG-G alternative surface.

In some embodiments, methods of making a library of FN3 polypeptides having a diversified C-CD-D-F-FG-G alternative surface comprising a diversified C beta-strand, CD loop, D beta-strand, F beta-strand, FG loop, or G beta-strand are provided, the method comprising providing a reference FN3 domain polypeptide having the amino acid sequence at least, or about, 80%, 81%, 82%, 83%, 84%, 85%, 86%, 87%, 88%, 89%, 90%, 91%, 92%, 93%, 94%, 95%, 96%, 97%, 98%, 99%, identical to that of SEQ ID NO: 44; introducing diversity into the consensus FN3 domain polypeptide by mutating at least one residue in the C beta-strand, the CD loop, the D beta-strand, the F beta-strand, the FG loop, or the G beta-strand to form the FN3 domain library having the diversified C-CD-D-F-FG-G alternative surface.

In the methods of making the library described herein, 1, 2, 3, 4, 5, 6, 7, 8, 9, 10, 11, 12, 13, 14, 15, 16, or 17 residues in any one of the C beta-strand, the CD loop, the D beta-strand, the F beta-strand, the FG loop, or the G beta-strand of SEQ ID NO: 44 can be mutated or modified. In some embodiments, the mutation is an substitution, insertion or deletion.

In some embodiments, a library produced by the methods provided for herein is provided. Generation of the scaffold proteins, FN3 domains (polypeptides or modules) of the, is, for example, achieved at the nucleic acid level. The libraries of the FN3 domains having substituted codons at one or more specific residues can be synthesized for example using standard PCR cloning methods, or chemical gene synthesis according to methods described in U.S. Pat. Nos. 6,521,427 and 6,670,127. Codons can be randomized using well known methods, for example degenerate oligonucleotides matching the designed diversity, or using Kunkel mutagenesis Kunkel et al., Methods Enzymol. 154, 367-382, 1987).

Libraries can be randomized at chosen codons using a random or defined set of amino acids. For example, variants in the library having random substitutions can be generated using NNK codons, which encode all 20 naturally occurring amino acids. In other diversification schemes, DVK codons can be used to encode amino acids Ala, Trp, Tyr, Lys, Thr, Asn, Lys, Ser, Arg, Asp, Glu, Gly, and Cys. Alternatively, NNS codons can be used to give rise to all 20 amino acid residues and simultaneously reducing the frequency of stop codons. The codon designations are according to the well-known IUB code. In some embodiments, the diversification is done without including methionine and/or cysteine as an option at the mutated residues.

The FN3 domains as any other proteins are prone to a variety of physical and/or chemical instabilities, resulting in adverse effects on the downstream processing. For instance, physical and chemical instability may lead to aggregation, degradation, reduced product yield, loss of potency, increased potential for immunogenicity, molecular heterogeneity, and loss of activity. Thus, presence of possible instability-inducing residues and recognition sequences may be minimize during the design of the libraries. For example, surface exposed methionine and tryptophan may be oxidized in storage conditions, possibly leading to loss in the protein scaffold potency. Presence of asparagine, in addition to contributing to well-known N-glycosylation recognition sites (NXS/T) may be deamidated when followed by glycine, possibly generating heterogeneity (Robinson, Proc Natl Acad Sci US A, 99, 5283-5288, 2002). Some or all of these amino acids thus may or may not be omitted from the mix used to randomize selected position. Furthermore, cysteine and praline may be omitted to minimize disulphide bridge formation and disruption of beta sheets.

Libraries of FN3 domains with biased amino acid distribution at positions to be diversified can be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule.

Synthesis of oligonucleotides with selected nucleotide “degeneracy” at certain positions is well known in that art, for example the TRIM approach (Knappek et al., J Mal Biol 296, 57-86, 1999; Garrard & Renner, Gene 128,103-109, 1993). Such sets of nucleotides having certain codon sets can be synthesized using commercially available nucleotide or nucleoside reagents and apparatus.

Standard cloning and expression techniques are used to clone the libraries into a vector or synthesize double stranded cDNA cassettes of the library, to express, or to translate the libraries in vitro. For example, cis-display can be used to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Natl Acad Sci USA 101, 2806-2810, 2004). Other methods can be used, for example ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768). The libraries of protein scaffolds may be expressed as fusion proteins displayed on the surface for example of any suitable bacteriophage. Methods for displaying fusion polypeptides on the surface of a bacteriophage are well known (U.S. Pat. Pub. No. 2011/0118144; Int. Pat. Pub. No. WO2009/085462; U.S. Pat. Nos. 6,969,108; 6,172,197; 5,223,409; 6,582,915; 6,472,147).

### Screening

Screening engineered protein FN3 domains or libraries of FN3 domain variants for specific binding to target molecules can be achieved for example by producing the library using cis display as described in Examples and in Odegrip et al., Proc Natl Acad Sci US 101, 2806-2810, 2004, and assaying the library for specific binding to a target molecule by any method known in the art. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The FN3 domains can bind human or other mammalian proteins with a wide range of affinities (KD)—Typically a FN3 domain can bind to a target protein with a KD equal to or less than about 10-7 M, 10-8 M, 10-9 M, 10-10 M, 10-11 M, 10-12 M, 10-13 M, 10-14 M, or 10-15 M as determined by surface plasmon resonance or the Kinexa method, as practiced by those of skill in the art. The affinity of a FN3 domain for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular FN3 domain-antigen interaction can vary if measured under different conditions (e.g., osmolarity, pH). Thus, measurements of affinity and other antigen-binding parameters (e.g., KD, Kon, K0 ff) are preferably made with standardized solutions of protein scaffold and antigen, and a standardized buffer, such as the buffer described herein. Other screening methods are also described in U.S. Pat. Nos. 7,842,476 and 8,679,781, each of which is hereby incorporated by reference in its entirety.

### Nucleic Acid Molecules and Vectors

The disclosure provides for nucleic acids encoding the FN3 as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof. Certain exemplary polynucleotides are disclosed herein, however, other polynucleotides which, given the degeneracy of the genetic code or codon preferences in a given expression system, encode the protein scaffolds and libraries of the protein scaffolds disclosed herein are also within the scope.

The polynucleotides disclosed herein may be produced by chemical synthesis such as solid phase polynucleotide synthesis on an automated polynucleotide synthesizer and assembled into complete single or double stranded molecules. Alternatively, the polynucleotides disclosed herein may be produced by other techniques such a PCR followed by routine cloning. Techniques for producing or obtaining polynucleotides of a given known sequence are well known in the art.

The polynucleotides disclosed herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fe or bacteriophage coat protein such as pIX or pIII. An exemplary polynucleotide comprises sequences for a Tac promoter, sequences encoding the FN3 domain library and repA, cis element, and a bacterial origin of replication (ori). Another exemplary polynucleotide comprises a pelB or ompA signal sequence, pIII or pIX bacteriophage coat protein, FN3 domain, and a polyA site.

Another embodiment is a vector comprising at least one polynucleotide disclosed herein. Such vectors may be plasmid vectors, viral vectors, vectors for baculovirus expression, transposon based vectors or any other vector suitable for introduction of the polynucleotides into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

### Host Cell Selection or Host Cell Engineering

An FN3 domain disclosed herein can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2nd Edition, Cold Spring Harbor, NY (1989); Harlow and Lane, Antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

The host cell chosen for expression may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21 (DE3), BL21-GOLD (DE3), XLI-Blue, JM109, HMS1 74, HMS1 74 (DE3), and any of the natural or engineered E. coli spp, Klebsiella spp., or Pseudomonas spp strains.

### Uses of FN3 Domains

The compositions of the FN3 domain (module)-based molecules described herein and generated by any of the above described methods may be used to diagnose, monitor, modulate, treat, alleviate, help prevent the incidence of, or reduce the symptoms of human disease or specific pathologies in cells, tissues, organs, fluid, or, generally, a host. A FN3 domain engineered for a specific purpose may be used to treat an immune-mediated or immune-deficiency disease, a metabolic disease, a cardiovascular disorder or disease; a malignant disease; a neurologic disorder or disease; an infection such as a bacterial, viral or parasitic infection; or other known or specified related condition including swelling, pain, and tissue necrosis or fibrosis.

Such a method can comprise administering an effective amount of a composition or a pharmaceutical composition comprising at least one FN3 domain specifically binding a target molecule to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

The FN3 polypeptides can be linked to another therapeutic to facilitate delivery of the therapeutic. Thus, the FN3 polypeptides can be used to deliver a therapeutic to a cell expressing a target that the FN3 polypeptide binds to, such as CD71.

Pharmaceutical Compositions Comprising FN3 domain-based Proteins

The FN3 domains specifically binding target molecules which are modified or unmodified, monomers, dimers, or multimers, mono-, bi- or multi-specific, can be isolated using separation procedures well known in the art for capture, immobilization, partitioning, or sedimentation, and purified to the extent necessary for commercial applicability. They can also be conjugated with nucleic acid molecules or other therapeutics. In some embodiments, the nucleic acid molecule is a siRNA or antisense molecule.

For therapeutic use, the FN3 domains specifically binding a target molecule may be prepared as pharmaceutical compositions containing an effective amount of the FN3 domain as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21st Edition, Troy, D. B. ed., Lippincott Williams and Wilkins, Philadelphia, PA 2006, Part 5, Pharmaceutical Manufacturing pp 691-1092, See especially pp. 958-989.

The mode of administration for therapeutic use of the FN3 domains specifically binding a target molecule may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal); using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracelebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

### Enumerated Embodiments

Embodiments provided herein also include, but are not limited to:

While having described the embodiments in general terms, certain embodiments are further disclosed in the following examples that should not be construed as limiting the scope of the claims.

1. A library comprising a plurality of fibronectin type III module (FN3) domains (polypeptides) having a diversified C-CD-D-F-FG-G alternative surface comprising a diversified C beta-strand, a CD loop, a D beta-strand, an F beta-strand, an FG loop and a G beta-strand, wherein the polypeptides comprise an amino acid sequence of:

(SEQ ID NO: 44)
MLSPPSNLRVTDVTSTSVTLSWKPPAPITGYXVXYXEXXXXG
EWKXVXVPGSETSYTVTGLKPGTEYXFXVXAVNGAXXGXPSQ
XVXVTT

or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 44, wherein each X is, independently, any amino acid.

2. The library of embodiment 1, wherein each X is, independently, any amino acid, except a methionine or a cysteine.

3. The library of embodiments 1 or 2, wherein the polypeptides comprises at least one mutated amino acid residue as compared to SEQ ID NO: 24 in one or more of, or each of, the C beta-strand, the CD loop, the D beta-strand, the F beta-strand, the FG loop, or the G beta-strand to form the FN3 domain library having the diversified C-CD-D-F-FG-G alternative surface.

4. The library of any one of embodiment 1-3, wherein the plurality of polypeptides have one or more mutations (e.g. substitutions, insertions, or deletions) at a position that corresponds to positions 32, 34, 36, 38, 39, 40, 41, 46, 48, 68, 70, 72, 78, 79, 81, 85, and/or 87 of SEQ ID NO: 24.

5. The library of any one of embodiments 1-4, wherein the diversified C beta-strand has an amino acid sequence of TGYXVXYXE (SEQ ID NO: 45), wherein each X is, independently, any amino acid except a methionine or a cysteine.

6. The library of any one of embodiments 1-5, wherein the diversified CD loop has an amino acid sequence of XXXXGE (SEQ ID NO: 46), wherein each X is, independently, any amino acid except a methionine or a cysteine.

7. The library of any one of embodiments 1-6, wherein the diversified D beta-strand has an amino acid sequence of WKXVXVP (SEQ ID NO: 47), wherein each X is, independently, any amino acid except a methionine or a cysteine.

8. The library of any one of embodiments 1-7, wherein the diversified F beta-strand has an amino acid sequence of TEYXFXVXAV (SEQ ID NO: 48), wherein each X is, independently, any amino acid except a methionine or a cysteine.

9. The library of any one of embodiments 1-8, wherein the diversified FG loop has an amino acid sequence of NGAXXG (SEQ ID NO: 49), wherein each X is, independently, any amino acid except a methionine or a cysteine.

10. The library of any one of embodiments 1-9, wherein the diversified G beta-strand has an amino acid sequence XPSQXVXVTT (SEQ ID NO: 50), wherein each X is, independently, any amino acid except a methionine or a cysteine.

11. The library of any one of embodiments 1-10, wherein the library comprises an amino acid sequence having an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

12. The library of any one of embodiments 1-11, wherein:
the diversified C beta-strand has an amino acid sequence of TGYXVXYXE (SEQ ID NO: 45), wherein each X is, independently, any amino acid except a methionine or a cysteine;
the diversified CD loop has an amino acid sequence of XXXXGE (SEQ ID NO: 46), wherein each X is, independently, any amino acid except a methionine or a cysteine;
the diversified D beta-strand has an amino acid sequence of WKXVXVP (SEQ ID NO: 47), wherein each X is, independently, any amino acid except a methionine or a cysteine;
the diversified F beta-strand has an amino acid sequence of TEYXFXVXAV (SEQ ID NO: 48), wherein each X is, independently, any amino acid except a methionine or a cysteine;
the diversified FG loop has an amino acid sequence of NGAXXG (SEQ ID NO: 49), wherein each X is, independently, any amino acid except a methionine or a cysteine; and
wherein the diversified G beta-strand has an amino acid sequence XPSQXVXVTT (SEQ ID NO: 50), wherein each X is, independently, any amino acid except a methionine or a cysteine.

13. A method of producing the library of any one of embodiments 1-12, the method comprising expressing a polynucleotide encoding the plurality of polypeptides.

14. A method of making a library of fibronectin module of type III (FN3) domains having a diversified C-CD-F-FG-G alternative surface comprising a diversified one or more, or each of, C beta-strand, a CD loop, an F beta-strand, an FG loop and G-beta strand, comprising
a. providing a reference FN3 domain polypeptide having an amino acid sequence at least 80% identical to that of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44; and
b. introducing diversity into the reference FN3 domain polypeptide by mutating at least one residue in the C beta-strand, CD loop region, F beta-strand region, FG loop region, or G-beta strand region to form the FN3 domain library having the diversified C-CD-F-FG-G alternative surface, wherein:
the diversified C beta-strand has an amino acid sequence of TGYXVXYXE (SEQ ID NO: 45), wherein each X is, independently, any amino acid except a methionine or a cysteine;
the diversified CD loop has an amino acid sequence of XXXXGE (SEQ ID NO: 46), wherein each X is, independently, any amino acid except a methionine or a cysteine;
the diversified D beta-strand has an amino acid sequence of WKXVXVP (SEQ ID NO: 47), wherein each X is, independently, any amino acid except a methionine or a cysteine;
the diversified F beta-strand has an amino acid sequence of TEYXFXVXAV (SEQ ID NO: 48), wherein each X is, independently, is any amino acid except a methionine or a cysteine;
the diversified FG loop has an amino acid sequence of NGAXXG (SEQ ID NO: 49), wherein each X is, independently, any amino acid except a methionine or a cysteine; and
the diversified G beta-strand has an amino acid sequence XPSQXVXVTT (SEQ ID NO: 50), wherein each X is, independently, any amino acid except a methionine or a cysteine.

15. A library produced by the method of embodiments 13 or 14.

16. A method of obtaining a polypeptide comprising a fibronectin type III module (FN3) domain having a diversified C-CD-D-F-FG-G alternative surface that binds or specifically binds to a target molecule, comprising contacting the library of any one of embodiments 1-12 with the target molecule and isolating the polypeptide that binds or specifically binds to the target molecule.

17. A polypeptide having an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

18. A polypeptide comprising an amino acid sequence of:

(SEQ ID NO: 44)
MLSPPSNLRVTDVTSTSVTLSWKPPAPITGYXVXYXEXXXXG
EWKXVXVPGSETSYTVTGLKPGTEYXFXVXAVNGAXXGXPSQ
XVXVTT

wherein each X is, independently, any amino acid.

19. The polypeptide of embodiment 18, wherein each X is, independently, any amino acid, except methionine or cysteine.

20. A polypeptide comprising an amino acid sequence of:

(SEQ ID NO: 74)
LSPPSNLRVTDVTSTSVTLSWKPPAPITGYXVXYXEXXXXGE
WKXVXVPGSETSYTVTGLKPGTEYXFXVXAVNGAXXGXPSQX
VXVTT

wherein each X is, independently, any amino acid.

21. The polypeptide of embodiment 20, wherein each X is, independently, any amino acid, except methionine or cysteine.

22. A pharmaceutical composition comprising the polypeptide of embodiments 17-21.

23. A nucleic acid molecule encoding the polypeptide of embodiment 22.

24. A plurality of nucleic acid molecules encoding the library of polypeptides of any one of embodiments 1-12.

25. A host cell comprising the nucleic acid molecule of embodiment 24.

### EXAMPLES

### Example 1: Design of Fn3 Domain Human Consensus Sequence (HumCon)

The Prosite sequence database alignment of fibronectin type III domains (http://prosite.expasy.org/PDOC50853) was used as the starting point for generation of the consensus sequence. This alignment was edited to remove all non-human derived sequences, the resulting alignment contains 806 human sequences. The predominant residue at each of 94 positions was used to determine candidate consensus sequences. At two positions (47 and 89), two residues were equally predominant. These are N and T at position 47 and S and V at position 89. At every other position, a single residue is dominant. Because of the ambiguity at positions 47 and 89 four consensus sequences were generated (Table 3).

TABLE 3

| SEQ ID | Sequence |
|---|---|
| 1 | MPPSNLRVTDVTSTSVTLSWEPPEDGGGPITGYIVEYREADGSGEWQEVTVPGSE |
| | TSYTVTGLEPGTEYEFRVRAVNGAGEGPPSEPSPVTTPEP |
| 2 | MPPSNLRVTDVTSTSVTLSWEPPEDGGGPITGYIVEYREADGSGEWQEVTVPGSE |
| | TSYTVTGLEPGTEYEFRVRAVNGAGEGPPSEPVPVTTPEP |
| 3 | MPPSNLRVTDVTSTSVTLSWEPPEDGGGPITGYIVEYREADGSGEWQEVNVPGSE |
| | TSYTVTGLEPGTEYEFRVRAVNGAGEGPPSEPSPVTTPEP |
| 4 | MPPSNLRVTDVTSTSVTLSWEPPEDGGGPITGYIVEYREADGSGEWQEVNVPGSE |
| | TSYTVTGLEPGTEYEFRVRAVNGAGEGPPSEPVPVTTPEP |

### Loop Design:

Loop regions can be highly variable in both length and sequence. While 806 sequences were aligned to create the consensus and most sequences contain a residue at each of the 94 positions, some positions contained fewer sequences because of deletions, which typically occurred in loops. Thus, some loops could be shortened and result in a more stable sequence. The putative BC and CD loops were identified as regions of length variability. To test the hypothesis that these loops could be shortened, a series of deletions in these regions were designed using SEQ ID NO: 2 as a backbone sequence. The results are SEQ ID NOs: 5-13, listed below in Table 4.

TABLE 4

| SEQ ID | Sequence |
|---|---|
| 5 | MPPSNLRVTDVTSTSVTLSWEPPEDGGPITGYIVEYREADGSGEWQEVTVPGSETSYTVTG |
| | LEPGTEYEFRVRAVNGAGEGPPSEPVPVTTPEP |
| 6 | MPPSNLRVTDVTSTSVTLSWEPPEGGPITGYIVEYREADGSGEWQEVTVPGSETSYTVTGL |
| | EPGTEYEFRVRAVNGAGEGPPSEPVPVTTPEP |
| 7 | MPPSNLRVTDVTSTSVTLSWEPPGGGPITGYIVEYREADGSGEWQEVTVPGSETSYTVTGL |
| | EPGTEYEFRVRAVNGAGEGPPSEPVPVTTPEP |
| 8 | MPPSNLRVTDVTSTSVTLSWEPPGGPITGYIVEYREADGSGEWQEVTVPGSETSYTVTGLE |
| | PGTEYEFRVRAVNGAGEGPPSEPVPVTTPEP |
| 9 | MPPSNLRVTDVTSTSVTLSWEPPGPITGYIVEYREADGSGEWQEVTVPGSETSYTVTGLEP |
| | GTEYEFRVRAVNGAGEGPPSEPVPVTTPEP |
| 10 | MPPSNLRVTDVTSTSVTLSWEPGPITGYIVEYREADGSGEWQEVTVPGSETSYTVTGLEPG |
| | TEYEFRVRAVNGAGEGPPSEPVPVTTPEP |
| 11 | MPPSNLRVTDVTSTSVTLSWEPPEDGGGPITGYIVEYREAGSGEWQEVTVPGSETSYTVTG |
| | LEPGTEYEFRVRAVNGAGEGPPSEPVPVTTPEP |
| 12 | MPPSNLRVTDVTSTSVTLSWEPPEDGGGPITGYIVEYREASGEWQEVTVPGSETSYTVTGL |
| | EPGTEYEFRVRAVNGAGEGPPSEPVPVTTPEP |
| 13 | MPPSNLRVTDVTSTSVTLSWEPPEDGGGPITGYIVEYRESGEWQEVTVPGSETSYTVTGLE |
| | PGTEYEFRVRAVNGAGEGPPSEPVPVTTPEP |

### Gene Synthesis, Expression and Characterization

Genes for the thirteen sequences from Tables 3 and 4 were designed with a C-terminal His tag and cloned into an expression vector under control of the T5 promoter. Expression yields as mg/L of culture were assessed in shake flasks and melting temperatures were assessed by differential scanning calorimetry (Table 5). An SDS PAGE assay was established to confirm monomeric homogeneity using MW standards. Some clones had behavior consistent with dimer formation hypothesized to be due to strand swapping that could be demonstrated by SDS PAGE and size exclusion chromatography.

TABLE 5

| SEQ | | Expression | Tm (° C.) pH | Pass |
|---|---|---|---|---|
| ID | Gene Name | yield (mg/L) | 7.5/4.5 | SDS page |
| 1 | HumCon SEQ1 | 79 | 85.7/98 | No |
| 2 | HumCon SEQ2 | 55 | 94.4 | No |
| 3 | HumCon SEQ3 | 56 | 84.6 | No |
| 4 | HumCon SEQ4 | 49 | 93.5 | No |
| 5 | HumCon SEQ2a | 50 | 96.6 | No |
| 6 | HumCon SEQ2b | 41 | 95.8 | ND |
| 7 | HumCon SEQ2c | 40 | 94.6 | ND |
| 8 | HumCon SEQ2d | 46 | 94.9 | ND |
| 9 | HumCon SEQ2e | 44 | 89.3 | ND |
| 10 | HumCon SEQ2f | 49 | 84.7 | ND |
| 11 | HumCon SEQ2g | 35 | 97.4 | No |
| 12 | HumCon SEQ2h | 70 | 94.2 | ND |
| 13 | HumCon SEQ2i | 95 | 85.7 | ND |

### Additional Optimization (N-Terminus, Prolines, pI)

SEQ ID NO: 2 was chosen as the lead candidate to engineer for improved biophysical properties. A series of mutations were designed to 1) remove prolines from segments anticipated to have beta sheet structure based on homology modeling to other FN3 domain structures with the goal of minimizing strand swapping; or 2) increase predicted pI values to enable simplified manufacturing and formulation properties. Sequences for these variants are listed in Table 6. Each protein was expressed in E coli, purified via a C-terminal His tag and assessed for solubility (expression of soluble protein/L E. coli), stability (Tm by differential scanning calorimetry) and monomeric homogeneity (SDS-PAGE) compared to the parent clone (SEQ ID NO: 2) (Table 7).

TABLE 6

| SEQ ID | Sequence |
|---|---|
| 14 | MLSPPSNLRVTDVTSTSVTLSWEPPEDGGGPITGYIVEYREADGSGEWQEVTVPGSETSYT |
| | VTGLEPGTEYEFRVRAVNGAGEGPPSEPVPVTTPEP |
| 15 | MSAPPSNLRVTDVTSTSVTLSWEPPEDGGGPITGYIVEYREADGSGEWQEVTVPGSETSYT |
| | VTGLEPGTEYEFRVRAVNGAGEGPPSEPVPVTTPEP |
| 16 | MPPSNLRVTDVTSTSVTLSWEPPEDGGGPITGYIVEYREADGSGEWQEVTVPGSETSYTVT |
| | GLEPGTEYEFRVRAVNGAGEGPPSEVTVTTPEP |
| 17 | MPPSNLRVTDVTSTSVTLSWEPPEDGGGPITGYIVEYREADGSGEWQEVTVPGSETSYTVT |
| | GLEPGTEYEFRVRAVNGAGEGPPSESVTVTTPEP |
| 18 | MLSPPSNLRVTDVTSTSVTLSWEPPEDGGGPITGYIVEYREADGSGEWQEVTVPGSETSYT |
| | VTGLEPGTEYEFRVRAVNGAGEGPPSESVTVTTPEP |
| 19 | MSAPPSNLRVTDVTSTSVTLSWEPPEDGGGPITGYIVEYREADGSGEWQEVTVPGSETSYT |
| | VTGLEPGTEYEFRVRAVNGAGEGPPSESVTVTTPEP |
| 20 | MLSPPSNLRVTDVTSTSVTLSWEPPEPITGYIVEYREADGSGEWQEVTVPGSETSYTVTGLE |
| | PGTEYEFRVRAVNGAGEGPPSESVTVTTPEP |
| 21 | MPPSNLRVTDVTSTSVTLSWEPPEPITGYIVEYREADGSGEWQEVTVPGSETSYTVTGLEP |
| | GTEYEFRVRAVNGAGEGPPSEVTVTTPEP |
| 22 | MLSPPSNLRVTDVTSTSVTLSWKPPAPITGYIVEYREKDGSGEWKEVTVPGSETSYTVTGLK |
| | PGTEYEFRVRAVNGAGEGPPSQSVTVTTPEP |
| 23 | MLSPPSNLRVTDVTSTSVTLSWKPPADGGGPITGYIVEYREKDGSGEWKEVTVPGSETSYT |
| | VTGLKPGTEYEFRVRAVNGAGEGPPSQSVTVTTPEP |
| 24 | MLSPPSNLRVTDVTSTSVTLSWKPPAPITGYIVEYREKDGSGEWKEVTVPGSETSYTVTGLK |
| | PGTEYEFRVRAVNGAGEGPPSQSVTVTT |

TABLE 7

| | | | Expression | | | Pass |
|---|---|---|---|---|---|---|
| SEQ | | | yield | Tm (° C.) | Tm (° C.) | SDS |
| ID | Gene Name | pI | (mg/L) | at pH 7.4 | at pH 4.5 | page |
| 14 | HumCon | 3.9 | 50.1 | ND | ND | No |
| | SEQ2j | | | | | |
| 15 | HumCon | 3.9 | 4.6 | ND | ND | No |
| | SEQ2k | | | | | |
| 16 | HumCon | 3.9 | 113.1 | ND | ND | No |
| | SEQ2l | | | | | |
| 17 | HumCon | 3.9 | 109.7 | ND | ND | No |
| | SEQ2m | | | | | |
| 18 | HumCon | 3.9 | 72.7 | ND | ND | No |
| | SEQ2n | | | | | |
| 19 | HumCon | 3.9 | 60.1 | ND | ND | No |
| | SEQ2o | | | | | |
| 20 | HumCon | 4.0 | 162.1 | ND | ND | No |
| | SEQ2p | | | | | |
| 21 | HumCon | 4.0 | 215.2 | ND | ND | No |
| | SEQ2q | | | | | |
| 22 | HumCon | 4.8 | 218.8 | 99.4 | 100.2 | Yes |
| | SEQ2r | | | | | |
| 23 | HumCon | 4.7 | 120.1 | ND | ND | No |
| | SEQ2s | | | | | |
| 24 | HumCon | 5.0 | 230.2 | 97.0 | 97.1 | Yes |
| | SEQ2t | | | | | |

### Library Design-Alanine Scanning

To test the viability of the library design strategy, nineteen mutant proteins were designed wherein each variant protein encoded one alanine residue at a unique position intended to be part of designed binding interface. The alanine scanning mutant sequences are listed in Table 8. Each protein was expressed in E coli, purified via a C-terminal His tag and characterized to ensure the biophysical properties were consistent with those for the parental clone (SEQ ID NO: 24). Protein variants were assessed for solubility (expression of soluble protein/L), stability (Tm by differential scanning calorimetry) and monomeric homogeneity (SDS-PAGE) (Table 9).

TABLE 8

| SEQ ID | Sequence |
|---|---|
| 25 | MLSPPSNLRVTDVTSTSVTLSWKPPAPITGYAVEYREKDGSGEWKE |
| | VTVPGSETSYTVTGLKPGTEYEFRVRAVNGAGEGPPSQSVTVTT |
| 26 | MLSPPSNLRVTDVTSTSVTLSWKPPAPITGYIVAYREKDGSGEWKE |
| | VTVPGSETSYTVTGLKPGTEYEFRVRAVNGAGEGPPSQSVTVTT |
| 27 | MLSPPSNLRVTDVTSTSVTLSWKPPAPITGYIVEYAEKDGSGEWKE |
| | VTVPGSETSYTVTGLKPGTEYEFRVRAVNGAGEGPPSQSVTVTT |
| 28 | MLSPPSNLRVTDVTSTSVTLSWKPPAPITGYIVEYREADGSGEWKE |
| | VTVPGSETSYTVTGLKPGTEYEFRVRAVNGAGEGPPSQSVTVTT |
| 29 | MLSPPSNLRVTDVTSTSVTLSWKPPAPITGYIVEYREKAGSGEWKE |
| | VTVPGSETSYTVTGLKPGTEYEFRVRAVNGAGEGPPSQSVTVTT |
| 30 | MLSPPSNLRVTDVTSTSVTLSWKPPAPITGYIVEYREKDASGEWKE |
| | VTVPGSETSYTVTGLKPGTEYEFRVRAVNGAGEGPPSQSVTVTT |
| 31 | MLSPPSNLRVTDVTSTSVTLSWKPPAPITGYIVEYREKDGAGEWKE |
| | VTVPGSETSYTVTGLKPGTEYEFRVRAVNGAGEGPPSQSVTVTT |
| 32 | MLSPPSNLRVTDVTSTSVTLSWKPPAPITGYIVEYREKDGSGEWKA |
| | VTVPGSETSYTVTGLKPGTEYEFRVRAVNGAGEGPPSQSVTVTT |
| 33 | MLSPPSNLRVTDVTSTSVTLSWKPPAPITGYIVEYREKDGSGEWKE |
| | VAVPGSETSYTVTGLKPGTEYEFRVRAVNGAGEGPPSQSVTVTT |
| 34 | MLSPPSNLRVTDVTSTSVTLSWKPPAPITGYIVEYREKDGSGEWKE |
| | VTVPGSETSYTVTGLKPGTEYAFRVRAVNGAGEGPPSQSVTVTT |
| 35 | MLSPPSNLRVTDVTSTSVTLSWKPPAPITGYIVEYREKDGSGEWKE |
| | VTVPGSETSYTVTGLKPGTEYEFAVRAVNGAGEGPPSQSVTVTT |
| 36 | MLSPPSNLRVTDVTSTSVTLSWKPPAPITGYIVEYREKDGSGEWKE |
| | VTVPGSETSYTVTGLKPGTEYEFRVAAVNGAGEGPPSQSVTVTT |
| 37 | MLSPPSNLRVTDVTSTSVTLSWKPPAPITGYIVEYREKDGSGEWKE |
| | VTVPGSETSYTVTGLKPGTEYEFRVRAVNGAAEGPPSQSVTVTT |
| 38 | MLSPPSNLRVTDVTSTSVTLSWKPPAPITGYIVEYREKDGSGEWKE |
| | VTVPGSETSYTVTGLKPGTEYEFRVRAVNGAGAGPPSQSVTVTT |
| 39 | MLSPPSNLRVTDVTSTSVTLSWKPPAPITGYIVEYREKDGSGEWKE |
| | VTVPGSETSYTVTGLKPGTEYEFRVRAVNGAGEGAPSQSVTVTT |
| 40 | MLSPPSNLRVTDVTSTSVTLSWKPPAPITGYIVEYREKDGSGEWKE |
| | VTVPGSETSYTVTGLKPGTEYEFRVRAVNGAGEGPPSASVTVTT |
| 41 | MLSPPSNLRVTDVTSTSVTLSWKPPAPITGYIVEYREKDGSGEWKE |
| | VTVPGSETSYTVTGLKPGTEYEFRVRAVNGAGEGPPSQSATVTT |
| 42 | MLSPPSNLRVTDVTSTSVTLSWKPPAPITGYIVEYREKDGSGEWKE |
| | VTVPGSETSYTVTGLKPGTEYEFRVRAVNGAGEGPPSQAVTVTT |
| 43 | MLSPPSNLRVTDVTSTSVTLSWKPPAPITGYIVEYREKDGSGEWKE |
| | VTVPGSETSYTVTGLKPGTEYEFRVRAVNGAGEGPPSQSVAVTT |

TABLE 9

| SEQ | | Expression | Tm | Pass SDS |
|---|---|---|---|---|
| ID | Gene Name | Yield (mg/L) | (° C.) | page |
| 25 | Seq2T_Ala1 | 147.51 | 89.3 | Yes |
| 26 | Seq2T_Ala2 | 133.77 | 94.3 | Yes |
| 27 | Seq2T_Ala3 | 135.23 | 89 | Yes |
| 28 | Seq2T_Ala4 | 128.85 | 95.4 | Yes |
| 29 | Seq2T_Ala5 | 141.85 | 95.8 | Yes |
| 30 | Seq2T_Ala6 | 108.87 | 95.1 | Yes |
| 31 | Seq2T_Ala7 | 144.44 | 95.8 | Yes |
| 32 | Seq2T_Ala8 | 107.47 | 98.2 | Yes |
| 33 | Seq2T_Ala9 | 119.4 | 96.4 | Yes |
| 34 | Seq2T_Ala10 | 122.46 | 90 | Yes |
| 35 | Seq2T_Ala11 | 149.42 | 86.9 | Yes |
| 36 | Seq2T_Ala12 | 129.46 | 88.5 | Yes |
| 37 | Seq2T_Ala13 | 138.53 | 97.8 | Yes |
| 38 | Seq2T_Ala14 | 39.56 | 98.4 | Yes |
| 39 | Seq2T_Ala15 | 190.85 | 95.6 | Yes |
| 40 | Seq2T_Ala16 | 123.87 | 96.4 | Yes |
| 41 | Seq2T_Ala17 | 126.74 | 89.8 | Yes |
| 42 | Seq2T_Ala18 | 140.8 | 96.0 | Yes |
| 43 | Seq2T_Ala19 | 123.6 | 94.5 | Yes |

### Library Validation by Screening Specific Bindings to CD71

A library of HumCon variants was built using standard molecular biology methods wherein 17 of the positions confirmed by the alanine mutation experiments were mutated to 18 possible amino acid (all amino acids except methionine and cysteine). This library was cloned in to the CIS display vector and panned for binders to CD71 using methods described herein. Specific binders were determined by ELISA. Members of the library that successfully bound to the CD71 target are listed in Table 10.

TABLE 10

| SEQ ID | SEQUENCE |
|---|---|
| 51 | MLSPPSNLRVTDVTSTSVTLSWKPPAPITGYIVFYGESLSEGEWKTVNVPG |
| | SETSYTVTGLKPGTEYTFGVDAVNGATFGPPSQWVFVTT |
| 52 | MLSPPSNLRVTDVTSTSVTLSWKPPAPITGYAVLYTETYYEGEWKQVHVP |
| | GSETSYTVTGLKPGTEYDFRVSAVNGATIGTPSQYVIVTT |
| 53 | MLSPPSNLRVTDVTSTSVTLSWKPPAPITGYVVLYWEASVQGEWKWVLV |
| | PGSETSYTVTGLKPGTEYTFGVDAVNGATFGPPSQWVFVTT |
| 54 | MLSPPSNLRVTDVTSTSVTLSWKPPAPITGYRVGYVEHLGAGEWKFVWVP |
| | GSETSYTVTGLKPGTEYTFGVDAVNGATFGPPSQWVFVTT |
| 55 | MLSPPSNLRVTDVTSTSVTLSWKPPAPITGYWVKYEEGPEYGEWKTVHVP |
| | GSETSYTVTGLKPGTEYTFGVDAVNGATFGPPSQWVFVTT |
| 56 | MLSPPSNLRVTDVTSTSVTLSWKPPAPITGYPVHYAESDLKGEWKRVVVP |
| | GSETSYTVTGLKPGTEYVFIVQAVNGAVDGNPSQIVVVTT |
| 57 | MLSPPSNLRVTDVTSTSVTLSWKPPAPITGYVVVYQETIHPGEWKNVHVP |
| | GSETSYTVTGLKPGTEYWFGVDAVNGATFGPPSQWVFVTT |
| 58 | MLSPPSNLRVTDVTSTSVTLSWKPPAPITGYWVSYLESTFIGEWKWVHVP |
| | GSETSYTVTGLKPGTEYVFVVQAVNGAPFGGPSQHVVVTT |
| 59 | MLSPPSNLRVTDVTSTSVTLSWKPPAPITGYIVHYSEFIFVGEWKHVLVPGS |
| | ETSYTVTGLKPGTEYDFRVSAVNGATIGTPSQYVIVTT |
| 60 | MLSPPSNLRVTDVTSTSVTLSWKPPAPITGYFVLYKELLQDGEWKTVLVP |
| | GSETSYTVTGLKPGTEYPFPVWAVNGALAGFPSQFVEVTT |
| 61 | MLSPPSNLRVTDVTSTSVTLSWKPPAPITGYAVYYNETWFQGEWKHVVVP |
| | GSETSYTVTGLKPGTEYYFHVEAVNGANPGKPSQHVVVTT |
| 62 | MLSPPSNLRVTDVTSTSVTLSWKPPAPITGYTVGYIEHPLSGEWKIVYVPGS |
| | ETSYTVTGLKPGTEYTFGVDAVNGATFGPPSQWVFVTT |
| 63 | MLSPPSNLRVTDVTSTSVTLSWKPPAPITGYSVTYNETTLDGEWKQVSVPG |
| | SETSYTVTGLKPGTEYVFSVNAVNGAKEGEPSQWVVVTT |
| 64 | MLSPPSNLRVTDVTSTSVTLSWKPPAPITGYQVVYSESVGWGEWKQVKVP |
| | GSETSYTVTGLKPGTEYWFGVDAVNGATFGPPSQWVFVTT |
| 65 | MLSPPSNLRVTDVTSTSVTLSWKPPAPITGYGVSYEEVYGFGEWKHVYVP |
| | GSETSYTVTGLKPGTEYVFSVLAVNGATFGPPSQWVFVTT |
| 66 | MLSPPSNLRVTDVTSTSVTLSWKPPAPITGYYVNYYEGPSDGEWKYVKVP |
| | GSETSYTVTGLKPGTEYWFWVQAVNGASVGPPSQDVGVTT |
| 67 | MLSPPSNLRVTDVTSTSVTLSWKPPAPITGYYVRYSESVDYGEWKWVLVP |
| | GSETSYTVTGLKPGTEYTFGVDAVNGATFGPPSQWVFVTT |
| 68 | MLSPPSNLRVTDVTSTSVTLSWKPPAPITGYYVSYEEHPNDGEWKYVEVP |
| | GSETSYTVTGLKPGTEYTFGVDAVNGATFGPPSQWVFVTT |
| 69 | MLSPPSNLRVTDVTSTSVTLSWKPPAPITGYFVSYTEGLEQGEWKFVLVPG |
| | SETSYTVTGLKPGTEYTFGVDAVNGATFGPPSQWVFVTT |
| 70 | MLSPPSNLRVTDVTSTSVTLSWKPPAPITGYWVTYEESTSHGEWKFVWVP |
| | GSETSYTVTGLKPGTEYTFGVDAVNGATFGPPSQWVFVTT |
| 71 | MLSPPSNLRVTDVTSTSVTLSWKPPAPITGYGVLYQESLHDGEWKWVLVP |
| | GSETSYTVTGLKPGTEYTFGVDAVNGATFGPPSQWVFVTT |
| 72 | MLSPPSNLRVTDVTSTSVTLSWKPPAPITGYYVFYDETVFLGEWKHVYVP |
| | GSETSYTVTGLKPGTEYAFTVAAVNGAVQGNPSQGVVVTT |
| 73 | MLSPPSNLRVTDVTSTSVTLSWKPPAPITGYYVQYNETTLTGEWKQVRVP |
| | GSETSYTVTGLKPGTEYIFNVHAVNGAHYGDPSQVVTVTT |

The present examples and embodiments provided for herein demonstrate that the FN3 domains provided for herein can be generated and used to a generate a library to produce molecules that can bind to a target protein of interest. These results could not have been predicted, and thus, a library of molecules, compositions comprising the same, and methods of using the same are provided for herein in a manner that could not have been predicted. It will be clear that the embodiments can be practiced otherwise than as particularly described in the foregoing description and examples. Numerous modifications and variations of the present embodiments are possible in light of the above teachings and, therefore, are within the scope of the appended claims.

1. A library comprising a plurality of fibronectin type III module (FN3) domains having a diversified C-CD-D-F-FG-G alternative surface comprising a diversified C beta-strand, a CD loop, a D beta-strand, an F beta-strand, an FG loop and a G beta-strand, wherein the polypeptides comprise an amino acid sequence of:
(SEQ ID NO: 44)
MLSPPSNLRVTDVISTSVTLSWKPPAPITGYXVXYXEXXXXGEW
KXVXVPGSETSYTVTGLKPGTEYXFXVXAVNGAXXGXPSQXVXV
TT
or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 44, wherein each X is, independently, any amino acid.

2. The library of claim 1, wherein each X is, independently, any amino acid, except a methionine or a cysteine.

3. The library of claim 1, wherein the polypeptides comprise at least one mutated amino acid residue as compared to SEQ ID NO: 24 in one or more of, or each of, the C beta-strand, the CD loop, the D beta-strand, the F beta-strand, the FG loop, or the G beta-strand to form the FN3 domain library having the diversified C-CD-D-F-FG-G alternative surface.

4. The library of claim 1, wherein the plurality of polypeptides have one or more mutations at a position that corresponds to positions 32, 34, 36, 38, 39, 40, 41, 46, 48, 68, 70, 72, 78, 79, 81, 85, and/or 87 of SEQ ID NO: 24.

5. The library of claim 1, wherein the diversified C beta-strand has an amino acid sequence of TGYXVXYXE (SEQ ID NO: 45), wherein each X is, independently, any amino acid except a methionine or a cysteine.

6. The library of claim 1, wherein the diversified CD loop has an amino acid sequence of XXXXGE (SEQ ID NO: 46), wherein each X is, independently, any amino acid except a methionine or a cysteine.

7. The library of claim 1, wherein the diversified D beta-strand has an amino acid sequence of WKXVXVP (SEQ ID NO: 47), wherein each X is, independently, any amino acid except a methionine or a cysteine.

8. The library of claim 1, wherein the diversified F beta-strand has an amino acid sequence of TEYXFXVXAV (SEQ ID NO: 48), wherein each X is, independently, any amino acid except a methionine or a cysteine.

9. The library of claim 1, wherein the diversified FG loop has an amino acid sequence of NGAXXG (SEQ ID NO: 49), wherein each X is, independently, any amino acid except a methionine or a cysteine.

10. The library of claim 1, wherein the diversified G beta-strand has an amino acid sequence XPSQXVXVTT (SEQ ID NO: 50), wherein each X is, independently, any amino acid except a methionine or a cysteine.

11. The library of claim 1, wherein the library comprises an amino acid sequence having an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

12. The library of claim 1, wherein:
the diversified C beta-strand has an amino acid sequence of TGYXVXYXE (SEQ ID NO: 45), wherein each X is, independently, any amino acid except a methionine or a cysteine;
the diversified CD loop has an amino acid sequence of XXXXGE (SEQ ID NO: 46), wherein each X is, independently, any amino acid except a methionine or a cysteine;
the diversified D beta-strand has an amino acid sequence of WKXVXVP (SEQ ID NO: 47), wherein each X is, independently, any amino acid except a methionine or a cysteine;
the diversified F beta-strand has an amino acid sequence of TEYXFXVXAV (SEQ ID NO: 48), wherein each X is, independently, any amino acid except a methionine or a cysteine;
the diversified FG loop has an amino acid sequence of NGAXXG (SEQ ID NO: 49), wherein each X is, independently, any amino acid except a methionine or a cysteine; and
wherein the diversified G beta-strand has an amino acid sequence XPSQXVXVTT (SEQ ID NO: 50), wherein each X is, independently, any amino acid except a methionine or a cysteine.

13. A method of producing the library of claim 1, the method comprising expressing a polynucleotide encoding the plurality of polypeptides.

14. A method of making a library of fibronectin module of type III (FN3) domains having a diversified C-CD-F-FG-G alternative surface comprising a diversified one or more, or each of, C beta-strand, a CD loop, an F beta-strand, an FG loop and G-beta strand, comprising
a. providing a reference FN3 domain polypeptide having an amino acid sequence at least 80% identical to that of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44; and
b. introducing diversity into the reference FN3 domain polypeptide by mutating at least one residue in the C beta-strand, CD loop region, F beta-strand region, FG loop region, or G-beta strand region to form the FN3 domain library having the diversified C-CD-F-FG-G alternative surface, wherein:
the diversified C beta-strand has an amino acid sequence of TGYXVXYXE (SEQ ID NO: 45), wherein each X is, independently, any amino acid except a methionine or a cysteine;
the diversified CD loop has an amino acid sequence of XXXXGE (SEQ ID NO: 46), wherein each X is, independently, any amino acid except a methionine or a cysteine;
the diversified D beta-strand has an amino acid sequence of WKXVXVP (SEQ ID NO: 47), wherein each X is, independently, any amino acid except a methionine or a cysteine;
the diversified F beta-strand has an amino acid sequence of TEYXFXVXAV (SEQ ID NO: 48), wherein each X is, independently, is any amino acid except a methionine or a cysteine;
the diversified FG loop has an amino acid sequence of NGAXXG (SEQ ID NO: 49), wherein each X is, independently, any amino acid except a methionine or a cysteine; and
the diversified G beta-strand has an amino acid sequence XPSQXVXVTT (SEQ ID NO: 50), wherein each X is, independently, any amino acid except a methionine or a cysteine.

15. (canceled)

16. A method of obtaining a polypeptide comprising a fibronectin type III module (FN3) domain having a diversified C-CD-D-F-FG-G alternative surface that binds or specifically binds to a target molecule, comprising contacting the library of claim 1 with the target molecule and isolating the polypeptide that binds or specifically binds to the target molecule.

17. A polypeptide having an amino acid sequence that is at least, or about, 80%, 81%, 82%, 83%, 84%, 85%, 86%, 87%, 88%, 89%, 90%, 91%, 92%, 93%, 94%, 95%, 96%, 97%, 98%, 99%, or is identical to a sequence selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

18. A polypeptide comprising an amino acid sequence of:
(SEQ ID NO: 44)
MLSPPSNLRVTDVTSTSVTLSWKPPAPITGYXVXYXEXXXXGEW
KXVXVPGSETSYTVTGLKPGTEYXFXVXAVNGAXXGXPSQXVXV
TT
wherein each X is, independently, any amino acid.

19. The polypeptide of claim 18, wherein each X is, independently, any amino acid, except methionine or cysteine.

20. A polypeptide comprising an amino acid sequence of:
(SEQ ID NO: 74)
LSPPSNLRVTDVTSTSVTLSWKPPAPITGYXVXYXEXXXXGEWK
XVXVPGSETSYTVTGLKPGTEYXFXVXAVNGAXXGXPSQXVXVI
T
wherein each X is, independently, any amino acid.

21. The polypeptide of claim 20, wherein each X is, independently, any amino acid, except methionine or cysteine.

22.-25. (canceled)
